(19) United States
(12) Patent Application Publication
(10) Pub. No.: US 2003/0108581 A1
(43) Pub. Date: Jun. 12, 2003

(54) Applicator and method for applying lanolin to a sore nipple

(76) Inventor: Brian H. Silver, Cary, IL (US)

Correspondence Address: Steven B. Courtright, BANIAK PINE & GANNON, 150 N. Wacker Drive, Suite 1200, Chicago, IL, 60606, US

(21) Appl. No.: 10/284,915

(22) Filed: Oct. 31, 2002

Related U.S. Application Data
(63) Continuation-in-part of application No. 09/853,966, filed on May 11, 2001.

(51) Int. Cl. A61K 7/00; A61F 13/00

(52) U.S. Cl. 424/401; 401/292

(57) ABSTRACT

A lanolin application kit comprising lanolin and a lanolin applicator is used to apply lanolin to a body surface, particularly on a nipple, without leaving undesired lanolin residue on the user, and so that contamination of other unused lanolin is minimized.

This is a continuation-in-part of application Ser. No. 09/853,966, filed May 11, 2001.

### BACKGROUND OF THE INVENTION

During breast-feeding, the nipple may crack, bleed, have a burning sensation or become sore. It is well-known that applying lanolin to the nipple may relieve such discomfort. Heretofore, lanolin was applied by dispensing the lanolin on one or more fingers of a person administering the lanolin, then using the finger(s) to apply the lanolin to the nipples. However, applying lanolin in such a manner may leave an undesired residue of lanolin on the person administering the lanolin. Moreover, having direct contact between the person administering the lanolin and the source of lanolin may result in contamination of the unused portion of lanolin. The present invention provides a method and applicator for applying the lanolin in a manner which overcomes these shortcomings, as well as providing further advantages as will become evident from the discussion that follows.

### SUMMARY OF THE INVENTION

It is a principal objective of the invention to provide a lanolin application kit comprising lanolin and a lanolin applicator which is used to apply lanolin to a skin surface, particularly on a nipple, without leaving undesired lanolin residue on the user, and in a manner that minimizes contamination of any unused lanolin. In one embodiment of the invention, lanolin is applied from a separate container to a sheet-like applicator, with the applicator sized to be placed over the nipple and adjacent breast and thereby temporarily positioned for treatment. The applicator need not be left in place, however, and therefore may take other sizes and shapes. The sheet itself may include a concavity or some other design adapted to better conform to the nipple. In alternate embodiments, the sheet may be a single layer of material, a multi-layered material, a pad, a bra-pad or formed of a material having absorbent qualities on a first side and moisture-resistant qualities on a second side, for example.

In another embodiment, the lanolin is provided for the user pre-applied to sheet-like applicators in a stack of sheets, pads, or bra pads. Each sheet or pad is separated from the stack and the lanolin either rubbed on the nipple/breast using the sheet, or simply put in place on the nipple/breast for treatment. Alternatively the lanolin-covered sheet could be used to apply the lanolin to the interior surface of a breast shield of a breast pump.

Various containers for the sheets with lanolin pre-applied thereon are disclosed, including a nesting arrangement of separators which serve to provide a storage container for a plurality of sheets. The sheets can be individual, or in pairs, for instance. Another application device has a plurality of sheets in a pad. Sheets with lanolin on one face thereof can be arranged in facial engagement in pairs in the stack, for instance. In another embodiment, single or paired sheets can be separately wrapped, and provided in a stack, boxed or the like. In another embodiment, the application device is a bra pad, which may inserted into a bra, e.g., a nursing bra or the like, adjacent the breast and positioned in contact with the nipple area.

These and other advantages and objectives of the present invention will be further understood upon consideration of the following detailed description of the invention taken in conjunction with the accompanying drawings, in which:

### BRIEF DESCRIPTION OF THE DRAWINGS

FIG. 1 shows an applicator container comprising stackable dividers that has been pulled apart to show applicator sheets with lanolin pre-dispensed thereon;

FIG. 2 shows a pad of sheets and its container;

FIG. 3 shows a lanolin tube and applicator;

FIG. 4 shows a modified applicator sheet; and

FIG. 5 shows an embodiment having paired sheets in a wrapper.

### DETAILED DESCRIPTION OF EMBODIMENTS OF THE INVENTION

One embodiment of the invention is directed in part to a kit and method of applying lanolin on a skin surface, preferably on a nipple, without leaving an undesirable lanolin residue on the person administering the lanolin. The method in one aspect comprises dispensing lanolin on a sheet-like applicator, then using the applicator to apply the lanolin on the desired skin surface. The applicator can also advantageously simply be placed over the nipple and left there for treatment. In the context of this invention, lanolin is dispensed on the applicator when it is put on the applicator or when the applicator is used to swab up an amount of lanolin to be used.

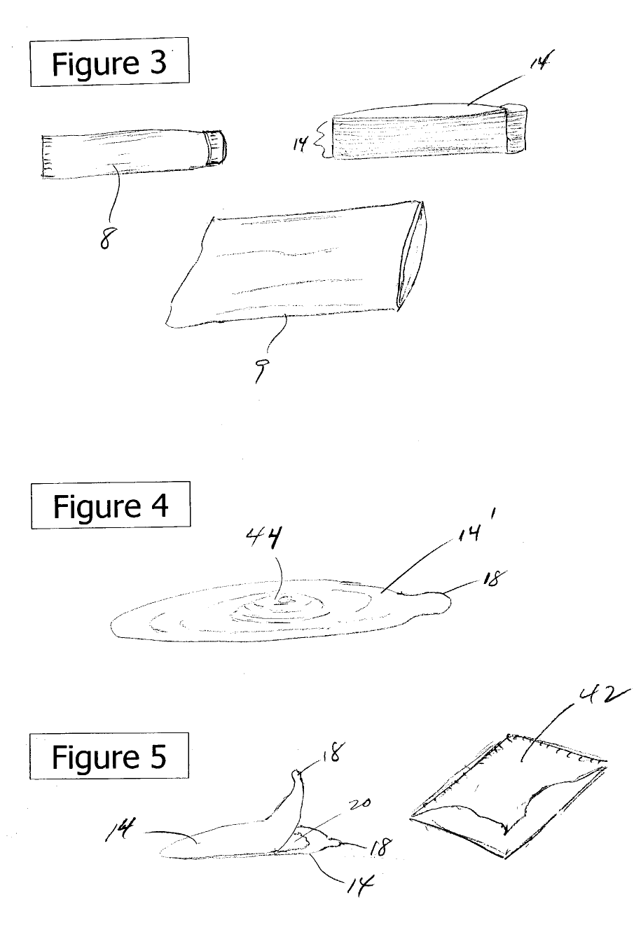

One embodiment provides a kit containing lanolin and the applicator, and optional directions that instruct the kit user on how to dispense the lanolin on the applicator and apply the lanolin to the desired skin surface. For instance, the kit could have a tube of lanolin 8 with a plurality of applicator sheets 14 (FIG. 3). The kit could be included in the ubiquitous reclosable plastic bag 9, or some other container.

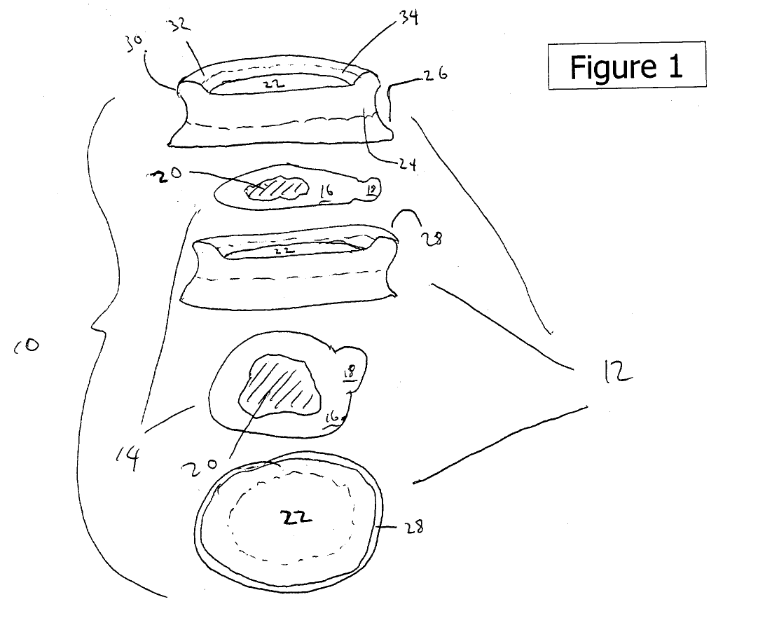

Another embodiment of the invention is shown in FIG. 1. The lanolin application kit 10 comprises separators or dividers 12 that are capable of frictionally engaging each other, top to bottom, with sufficient gripping force to define a container with sufficient space between the separators to store an applicator sheet 14. Sheet 14 comprises a body 16 and a gripping flange 18. In the preferred embodiment shown, lanolin 20 is provided on sheet 14 prior to storage.

Separator 12 includes a top surface 22 that is substantially horizontal and substantially planar. An engaging recess 24 is formed about the outer periphery 26 of separator 12. Recess 24 is defined by outer wall 30, top wall 32 and an inner wall 34, such that the three walls also define a rim 28, sized to match opposing recess 24. In this preferred embodiment, two separators 12 are capable of stacking on top of each other, such that rim 28 matingly engages recess 24 to provide the frictional grip (a plug fit) required to form a container for a sheet 14.

In the embodiment shown in FIG. 1, to access the applicator, the user applies an amount of force sufficient to disengage two separators. Then, the user grasps an applicator by flange 18, and applies the lanolin 20 on the desired skin surface. The sheet can be put in place on the nipple and breast. Alternatively, once sheet 14 is used, both the used sheet and its separator 12 can be thrown away.

As shown, several sheets can be individually stored by stacking the separators on top of each other. It will be noted that while a single sheet 14 is stored between adjacent separators, two sheets with their lanolin-covered surfaces in facial engagement could likewise be provided. A single sheet could likewise be folded upon itself, i.e., in facial engagement with itself, with a plurality of such folded sheets then stacked.

While the recess is described as defined by three walls 30, 32 and 34, numerous embodiments may define the recess and rim. For example, the three walls may merge to form one rounded U-shaped wall. Further, the rim/recess need not be exactly the periphery, but should be sufficiently close to the periphery to define an inner space to store the sheet(s). Moreover, while the separators are shown as substantially circular, the separators can have numerous shapes (e.g., rectangular, etc.).

Preferably, separator 12 is a thermoformed or molded plastic material, more preferably polystyrene. The sheet material 14 is preferably made from a spunlaced fabric such as Dupont Sontara® laminated with a microporous polyethylene backing. This material provides a breathable barrier backing for in situ placement. Alternate fabrics and backing materials are readily available.

Another way of separating pairs of lanolin-covered (at least in part) sheets, would be to place the pair in a foil packet, with such packets then provided to the user in a stack or box. This is illustrated in FIG. 5, where two sheets 14 are placed in facial engagement with lanolin 20 essentially sandwiched therebetween. The paired sheets 14 are provided to the user in a packet 42, which can be of a tear-open type or the like. Packet 42 may be made of foil or plastic sheet material, for instance, with the lanolin-covered sheets sealed therein. Furthermore, multiple pairs of sheets 14 in facial engagement could be stacked in a pad-like arrangement, and provided in a packet, pouch, box, or the like.

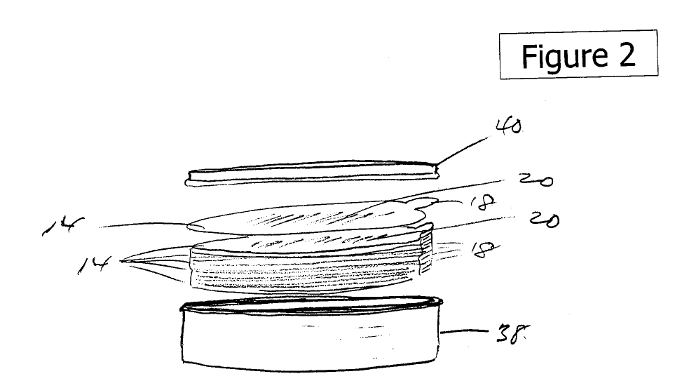

FIG. 2 illustrates yet another embodiment of the invention. Here, sheets 14 with lanolin 20 already deposited thereon on one side, are arranged in a stack. The stack can be held together by the inherent tackiness of the lanolin-coated sheets when placed in such facial engagement, or through a separate adhesion or other joinder of the sheets, such as a releasable glue or weak mechanical fixation, as of the gripping flanges 18.

The stack of sheets 14 constitutes a pad, and fits within a container having an appropriately sized receptacle base 38 and lid 40. Lid 40 snap-fits over the opening of the receptacle base, although many other kind of closures could be used. Moreover, other types of storage devices may be used, such as a foil-lined pouch, reclosable plastic bag, and so on. As used herein a pad may include two or more sheets.

A user of this second embodiment simply gains access to the stack of sheets 14, removes one from the stack, such as the topmost sheet 14, using the gripping flange 18, and then either rubs on the lanolin 20 as needed or simply places the sheet on the breast over the nipple with the lanolin against the breast/nipple. The lanolin could also be applied to lubricate a breastshield, by rubbing it onto the interior of the breastshield of a breast pump.

Referring to FIG. 4, a modified sheet 14′ is illustrated. This sheet 14′ has a concavity 44 formed therein, which is designed to better conform to the nipple and adjacent breast. Other ways to conform the applicator to the shape of the breast for when it is left in place are also available, such as one or more radial cuts in the sheet 14, just to name one.

While the present invention has been described in terms of particular embodiments, it will be understood that one skilled in the art can adopt other forms, arrangement, composition, materials and the like without departing from the spirit of this invention, yet still fall within the scope of the invention as hereafter set forth in claims that follow.

What is claimed is:

1. A lanolin application kit comprising:
a lanolin applicator having lanolin provided on at least one side thereof, said lanolin applicator further including a lanolin-free gripping part.

2. The lanolin application kit according to claim 1, wherein said lanolin applicator is provided in the form of one or more sheet.

3. The lanolin application kit according to claim 1, wherein said lanolin applicator is provided in the form of one or more pad.

4. The lanolin application kit according to claim 1, wherein said lanolin applicator is provided in the form of one or more bra pad.

5. The lanolin application kit according to claim 1, further comprising a container for storing said lanolin applicator.

6. The lanolin application kit according to claim 5 wherein said container includes a first separator and a second separator that is matingly engageable thereto to provide a space therebetween to store said lanolin applicator therein.

7. The lanolin application kit according to claim 6 wherein said first and second separators each include a substantially horizontal top surface having a recess and a rim, wherein said rim on said first separator is matingly engageable in said recess of said second separator.

8. The lanolin application kit of claim 1 wherein said lanolin applicator has a concavity for receiving the nipple therein.

9. The lanolin application kit of claim 1 wherein said lanolin applicator is adapted to conform to the general shape of the breast when placed thereon.

10. A method of applying lanolin to a human body surface comprising:
dispensing lanolin on an applicator; and
using said applicator to apply said lanolin to the human body surface.

11. The method according to claim 10 wherein the body surface is a nipple.

12. The method according to claim 11 wherein said applicator is a sheet sized to cover the nipple and some adjacent breast, and said sheet is placed over the nipple and left in place on the breast.

13. The method according to claim 10 wherein said applicator is a sheet and a plurality of sheets are provided, and further including the step of disposing lanolin on each of said plurality of sheets so that each said sheet is provided to the user with lanolin pre-applied.

14. The method of claim 13 wherein lanolin is disposed on one side of each said plurality of sheets, and adjacent pairs of said plurality of sheets are oriented with their lanolin-containing sides in facial engagement.

15. A lanolin application device for treating nipples, comprising:
a plurality of sheets, each sheet having a quantity of lanolin provided on a face thereof, said sheets being arranged in a pad.

16. The lanolin application device of claim 15 further comprising:
a container for said pad.

17. The lanolin application device of claim 15 wherein said sheets are each sized to be placed on a nipple and some surrounding breast.

18. The lanolin application device of claim 15 wherein each said sheet has a gripping portion which is free of lanolin.

19. The lanolin application device of claim 15 wherein each said sheet is folded upon itself with lanolin on at least part of said portions in facial engagement.

20. The lanolin application device of claim 15 wherein lanolin is disposed on one side of each sheet, and said sheets are further paired with their lanolin sides in facial engagement.

21. The lanolin application device of claim 15 wherein said sheet has a concavity for receiving the nipple therein.

22. The lanolin application device of claim 15 wherein said sheet is adapted to conform to the general shape of the breast when placed thereon.

23. A lanolin application device for treating nipples, comprising:
a plurality of sheets, each sheet having a quantity of lanolin provided on a face thereof; and
a container for said sheets, said container having a plurality of nesting dividers, said dividers separating one sheet from another.

24. A lanolin application device for treating nipples, comprising:
a plurality of sheets, each sheet having a quantity of lanolin provided on a face thereof; said sheets being organized in pairs which are in facial engagement; and
an arrangement for grouping said sheets, said arrangement having a plurality of dividing elements separating one pair of sheets from another.

25. A lanolin application device for treating nipples, comprising:
at least one application sheet having a quantity of lanolin provided on a face thereof, and a substantially lanolin-free gripping portion on said sheet.

26. The lanolin application device of claim 25 wherein a plurality of sheets are provided in a pad.

27. The lanolin application device of claim 25 wherein each sheet is individually enclosed in a package which is substantially lanolin impermeable.

28. The lanolin application device of claim 25 wherein a plurality of sheets are provided in a stack with pairs of sheets having their lanolin faces in facial engagement.

29. The lanolin application device of claim 25 wherein there are two sheets which are enclosed together in a package which is substantially lanolin impermeable.

30. The lanolin application device of claim 25 wherein said sheet has a concavity for receiving the nipple therein.

31. The lanolin application device of claim 25 wherein said sheet is adapted to conform to the general shape of the breast when placed thereon.

32. The lanolin application device of claim 25 wherein said sheet is sized to be placed on a nipple and some surrounding breast.

33. A lanolin application device for treating nipples comprising:
at least one application sheet having a quantity of lanolin provided on a face thereof, wherein said at least one application sheet is a bra pad having substantially lanolin-free portions on said face.

34. The lanolin application device according to claim 33, wherein said bra pad has a concavity for receiving the nipple therein.

35. A method of applying lanolin comprising:
providing at least one application sheet having a quantity of lanolin provided on a face thereof wherein said application sheet is a bra pad having substantially lanolin-free portions on said face, and
placing said application sheet on a nipple, then maintaining said bra pad in contact with the breast with a bra.
